(19) United States
(12) Patent Application Publication
(10) Pub. No.: US 2026/0263020 A1
(43) Pub. Date: Sep. 10, 2026

(54) SAFETY HOOK FOR CEILING MOUNTED MEDICAL IMAGING SYSTEM

(71) Applicant: GE Precision Healthcare LLC, Waukesha, WI (US)

(72) Inventors: Guoli Pan, Beijing (CN); Julien Marcotte, Saint Vrain (FR); Romain Chatelin, Plaisir (FR); Jinxin Xie, Beijing (CN); Weichao Hu, Beijing (CN)

(21) Appl. No.: 19/069,551

(22) Filed: Mar. 4, 2025

(51) Int. Cl. F16B 45/02

(52) U.S. Cl. CPC F16B 45/023

(57) ABSTRACT

A safety hook of a ceiling mount assembly operable to receive a load of a medical imaging system in a failure condition of the ceiling mount assembly is provided. The safety hook includes two parallel vertical holders having a first end operable to be attached to a body of the ceiling mount assembly and a second end having an aperture. A first of the two parallel vertical holders is configured to be positioned adjacent a first side of a mounting structure of the medical imaging system, and a second of the two parallel vertical holders is configured to be positioned adjacent a second side of the mounting structure of the medical imaging system. The safety hook includes a shaft extending through the apertures at the second end of the two parallel vertical holders and a slot hole in the mounting structure of the medical imaging system.

### FIELD

Certain embodiments relate to ceiling mounts for medical imaging systems. More specifically, certain embodiments relate to a safety hook for a ceiling mounted medical imaging system that prevents the ceiling mounted medical imaging system from falling if a screw or other attachment mechanism of the mounting assembly fails.

### BACKGROUND

Medical diagnostic imaging systems generate images of a patient through exposure to an energy source, such as X-rays passing through a patient, for example. The generated images may be used to provide a medical diagnosis of a patient, among other things. In some cases, the medical diagnostic imaging system may be mounted to a ceiling in an examination room. The medical diagnostic imaging system may be movable and rotatable to acquire medical image data from different positions and/or angles. One example of a ceiling mounted medical diagnostic imaging system is C-arm X-ray diagnostic equipment. The term C-arm generally refers to an X-ray imaging device having a rigid and/or articulating structural member having an X-ray source and an image detector assembly that are each located at an opposing end of the structural member so that the X-ray source and the image detector face each other. The structural member is typically “C” shaped and so is referred to as a C-arm. In this manner, X-rays emitted from the X-ray source can impinge on the image detector and provide an X-ray image of the object or objects that are placed between the X-ray source and the image detector. The weight of a C-arm X-ray diagnostic medical imaging system may be 800 kilograms or more. If a screw or other attachments mechanism of the mounting assembly were to loosen or break and the C-arm X-ray diagnostic medical imaging system were to fall on a patient, the result could be catastrophic.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY

A safety hook for a ceiling mounted medical imaging system that prevents the ceiling mounted medical imaging system from falling if a screw or other attachment mechanism of the mounting assembly fails is provided, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram illustrating components of an exemplary X-ray imaging system, in accordance with various embodiments.

FIG. 2 is a schematic diagram of a side view of an exemplary X-ray imaging system mounted to a ceiling, in accordance with various embodiments.

FIG. 3 is a schematic diagram of a lateral view of a lower ceiling mount configured to pivotally mount an X-ray imaging system to an upper ceiling mount installed on a ceiling, in accordance with various embodiments.

FIG. 4 is a schematic diagram of a lateral view of an X-ray imaging system mounted to a ceiling by a lower ceiling mount and an upper ceiling mount, in accordance with various embodiments.

FIG. 5 is a schematic diagram of a perspective view of an X-ray imaging system mounted to a ceiling by a lower ceiling mount and an upper ceiling mount, in accordance with various embodiments.

FIG. 6 is a schematic diagram of a cross-sectional view of a lower ceiling mount configured to pivotally mount an X-ray imaging system to an upper ceiling mount installed on a ceiling, in accordance with various embodiments.

FIG. 7 is a schematic diagram of an exploded view of a lower ceiling mount configured to pivotally mount an X-ray imaging system to an upper ceiling mount installed on a ceiling, in accordance with various embodiments.

FIG. 8 is a schematic diagram of a perspective view of a portion of an X-ray imaging system having a slot hole for receiving a safety hook, in accordance with various embodiments.

FIG. 9 is a schematic diagram of a perspective view of a safety hook provided between a lower ceiling mount and an X-ray imaging system, in accordance with various embodiments.

FIG. 10 is a schematic diagram of a cross-sectional view of a safety hook provided between a lower ceiling mount and an X-ray imaging system, in accordance with various embodiments.

### DETAILED DESCRIPTION

Certain embodiments may be found in a safety hook for a ceiling mounted medical imaging system that prevents the ceiling mounted medical imaging system from falling if a screw or other attachment mechanism of the mounting assembly fails. For example, aspects of the present disclosure have the technical effect of ensuring the safety of patients and operators by substantially reducing and/or eliminating the risk of a ceiling mounted medical imaging system falling if a screw or other attachment mechanism of the mounting assembly fails. In various embodiments, the safety hook takes no load during normal operation. Instead, the safety hook carries the load only if a failure occurs.

The foregoing summary, as well as the following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general-purpose signal processor or a block of random-access memory, hard disk, or the like) or multiple pieces of hardware. Similarly, the programs may be standalone programs, may be incorporated as subroutines in an operating system, may be functions in an installed software package, and the like. It should be understood that the various embodiments are not limited to the arrangements and instrumentality shown in the drawings. It should also be understood that the embodiments may be combined, or that other embodiments may be utilized, and that structural, logical and electrical changes may be made without departing from the scope of the various embodiments. The following detailed description is, therefore, not to be taken in a limiting sense, and the scope of the present disclosure is defined by the appended claims and their equivalents.

As used herein, an element or step recited in the singular and preceded with the word “a” or “an” should be understood as not excluding plural of said elements or steps, unless such exclusion is explicitly stated. Furthermore, references to “an exemplary embodiment,” “various embodiments,” “certain embodiments,” “a representative embodiment,” and the like are not intended to be interpreted as excluding the existence of additional embodiments that also incorporate the recited features. Moreover, unless explicitly stated to the contrary, embodiments “comprising”, “including”, or “having” an element or a plurality of elements having a particular property may include additional elements not having that property.

Also as used herein, the term “image” broadly refers to both viewable images and data representing a viewable image. However, many embodiments generate (or are configured to generate) at least one viewable image.

Furthermore, the term processor or processing unit, as used herein, refers to any type of processing unit that can carry out the required calculations needed for the various embodiments, such as single or multi-core Central Processing Unit (CPU), Accelerated Processing Unit (APU), Graphic Processing Unit (GPU), Digital Signal Processor (DSP), Field Programmable Gate Array (FPGA), System on a Chip (SoC), Application-Specific Integrated Circuit (ASIC), or a combination thereof.

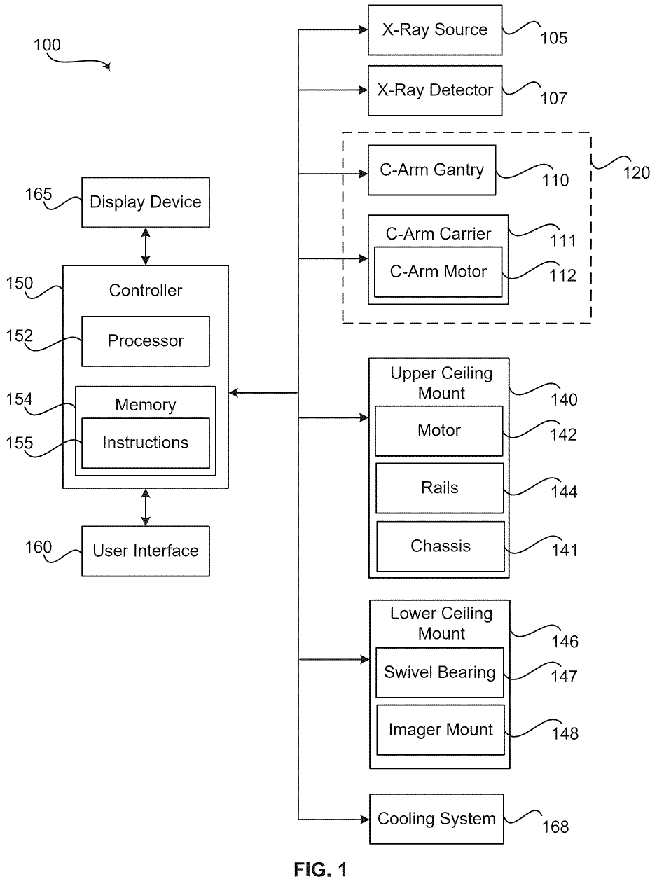

FIG. 1 is a block diagram illustrating components of an exemplary X-ray imaging system 100, in accordance with various embodiments. Referring to FIG. 1, there is shown an X-ray imaging system 100 that comprises a controller 150, a user interface 160, a display device 165, an X-ray source 105, and X-ray detector 107, a gantry structure 120, an upper ceiling mount 140, a lower ceiling mount 146, and a cooling system 168, among other things. In certain embodiments, the X-ray imaging system 100 is configured to perform vascular imaging, such as digital subtraction angiography (DSA). The X-ray imaging system 100 is configured to be mounted to a ceiling. In certain embodiments, the X-ray imaging system 100 is mounted to the ceiling at a fixed location. In certain embodiments, the X-ray imaging system 100 is mounted to the ceiling but may move along the ceiling (e.g., via chassis that moves along a rail system). The X-ray imaging system 100 includes an X-ray source 105 (or X-ray radiation source) and an X-ray detector 107 mounted on a C-arm gantry 110 (e.g., C-arm). In certain embodiments, the C-arm gantry 110 may be made of carbon fiber.

The C-arm gantry 110 is part of a gantry structure 120. The gantry structure 120 includes a C-arm motor 112 for adjusting the position of the C-arm gantry 110. More specifically, the C-arm gantry 110 is mechanically coupled to a C-arm carrier 111 (e.g., C-arm rotation device) which includes the C-arm motor 112, and the C-arm motor 112 may be driven to adjust the position of the C-arm gantry 110 with respect to the C-arm carrier 111. For example, the C-arm carrier 111 in conjunction with the C-arm motor 112 is configured to rotate the C-arm gantry 110 in an orbital direction relative to the C-arm carrier 111. In certain embodiments, the C-arm carrier 111 (via a motorized system) is configured to rotate a pivot (e.g., pivot point) where the C-arm carrier 111 is coupled to an end of an arm (e.g., L-arm) coupled or mounted to the ceiling. The C-arm carrier 111 rotates about a rotational axis (e.g., horizontal axis) of the pivot. In representative embodiments having an L-arm, the L-arm may rotate about a location where the other end of the L-arm (i.e., the end of the L-arm not connected to the pivot) is coupled to or mounted to the ceiling (via a lower ceiling mount 146). In various embodiments, the C-arm carrier 111 is coupled to or mounted to the ceiling (via the lower ceiling mount 146).

The upper ceiling mount 140 is coupled to a ceiling. In certain embodiments, the C-arm carrier 111 is coupled to the upper ceiling mount 140 via an L-arm (e.g., via the end of the L-arm not connected to the pivot) coupled to the lower ceiling mount 146. The upper ceiling mount 140 is configured to move (e.g., translocate) the X-ray imaging system 100 from one location to another location (e.g., in a linear direction) on the ceiling. The upper ceiling mount 140 includes a chassis 141. The upper ceiling mount 140 also includes a motor 142 and a rail system 144 (e.g., having rails). The rail system 144 is directly coupled to the ceiling. The motor 142 is configured to drive movement of the chassis 141 and, thus, the X-ray imaging system 100 along the rail system 144 (e.g., to adjust a position of the X-ray imaging system 100).

The lower ceiling mount 146 is configured to rotatably couple the gantry structure 120 to the upper ceiling mount 140. In various embodiment, the lower ceiling mount 146 may be operable to dampen vibrations that occur when the C-arm gantry 110 (e.g., made of carbon fiber) moves and/or deaccelerates (e.g., during the deacceleration phase) to a stop during movement of the gantry structure 120 along multiple axes or directions (e.g., combined directions). For example, the movement of the gantry structure 120 may include the rotational movement of the C-arm gantry 110 in the orbital direction, rotational movement about the axis of the pivot where the C-arm carrier 111 is coupled to an end of an arm (e.g., L-arm), and/or rotation about where an axis of where the lower ceiling mount 146 (e.g., axis of the swivel bearing 147) is coupled the ceiling. The lower ceiling mount 146 is configured to dampen vibrations caused by movement of the gantry structure 120 for an entirety of the gantry structure 120 along the multiple directions. The multiple directions may include orthogonal directions. For example, the orthogonal directions may include a vertical direction extending between a floor and the ceiling and a horizontal direction that is both perpendicular to the vertical direction and parallel with the floor.

The lower ceiling mount 146 includes a swivel bearing 147 and an imager mount 148. The imager mount 148 includes a body having a first end and a second end opposite the first end, a first mount connection disposed on the first end, and a second mount connection coupled to the second end. The imager mount 148 further comprises a safety hook that prevents the gantry structure 120 from falling if the first and/or second mount connections fail. The safety hook comprises two parallel vertical holders having a first end attached between the first end and the second end of the body, and a second end having an aperture. The two parallel vertical holders extend on opposite sides of a portion of the gantry structure 120 (e.g., a center rib of the L-arm). A shaft of the safety hook extends through the apertures in the second ends of the vertical holders and through a slot hole in the gantry structure 120. The shaft is secured to the vertical holders by a washer and nut, a clip ring, or any suitable attachment mechanism. The first mount connection and the second mount connection both directly contact both the gantry structure 120 and the body. In various embodiments, the safety hook directly contacts the body but does not receive a load of the gantry structure 120 in a normal condition. If the first and/or second mount connections fail, the safety hook receives the load of the gantry structure 120. In an exemplary embodiment, the first mount connection includes one or more mounting brackets, damping pads or dampers (e.g., made of elastomeric material such as rubber), and mounting screws and/or any suitable attachment mechanism. In certain embodiments, the second mount connection includes a shaft and bushings coupled to opposite ends of the shaft, and wherein the shaft extends through a portion of the gantry structure 120.

The swivel bearing 147 pivotally couples the gantry structure 120 to the upper ceiling mount 140 (e.g., the chassis) when the X-ray imaging system 100 is mounted to the ceiling. The swivel bearing 147 is disposed on a side of the body facing the ceiling. In certain embodiments, the body is made of a material that is stiffer than both the first mount connection and the second mount connection. The swivel bearing 147 is configured to enable the gantry structure 120 to rotate 360 degrees about a rotational axis of the swivel bearing 147.

The X-ray imaging system 100 further includes a controller 150 comprising suitable logic, circuitry, interfaces and/or code that may be operable to control operation of the X-ray imaging system 100. The controller 150 comprises a processor 152 and a non-transitory memory 154. A method for controlling the X-ray imaging system 100 may be stored as executable instructions 155 in the non-transitory memory 154 and executed by the processor 152.

The memory 154 may be one or more computer-readable memories integrated with the X-ray imaging system 100 and/or communicatively coupled (e.g., over a network) to the X-ray imaging system 100, such as a Picture Archiving and Communication System (PACS), a server, a hard disk, floppy disk, CD, CD-ROM, DVD, compact storage, flash memory, random access memory, read-only memory, electrically erasable and programmable read-only memory, optical storage medium, magnetic storage medium, solid-state storage medium, and/or any suitable memory. The memory 154 may include databases, libraries, sets of information, or other storage accessed by and/or incorporated with the processor 152, for example. The memory 154 may be able to store data temporarily or permanently, for example. The memory 154 may store processor-executable software code or instructions (e.g., firmware or software) 155, which are tangibly stored on a non-transitory computer readable medium. Additionally or alternatively, the memory 154 may store medical image data, data generated by the processor 152, and/or any suitable data.

The processor 152 may comprise suitable logic, circuitry, interfaces and/or code that may be operable to process medical image data for generating medical images (e.g., X-ray images) for presentation on a display device 165. The processor 152 is operable to perform one or more processing operations on the acquired medical image data. In an exemplary embodiment, the processor 152 may be operable to perform display processing and/or control processing, among other things. Acquired medical image data may be processed in real-time during an imaging examination as the medical image data is received. Additionally or alternatively, the medical image data may be stored temporarily during an imaging examination and processed in less than real-time in a live or off-line operation. In various embodiments, the processed image data can be presented at the display device 165 and/or may be stored at the memory 154. The memory 154 may be a local archive, a Picture Archiving and Communication System (PACS), or any suitable device for storing images and related information. The processor 152 may be one or more central processing units, graphic processing units, microprocessors, microcontrollers, and/or the like. The processor 152 may be an integrated component, or may be distributed across various locations, for example. In an exemplary embodiment, the processor 132 may be capable of receiving input information from a user interface 160 and/or memory 154, generating an output displayable by a display device 165, and manipulating the output in response to input information from a user interface 160, among other things. The processor 152 may be capable of executing any of the method(s) and/or set(s) of instructions 155 discussed herein in accordance with the various embodiments, for example.

The user interface 160 may be utilized to receive input from a user or operator of the X-ray imaging system 100. The user interface 160 may be communicatively coupled to the controller 150 for providing commands input by a user via the user interface 160 to the controller 150. For example, the user interface 160 may receive inputs of patient data, image acquisition parameters, settings, configuration parameters, select protocols and/or templates, and the like. In an exemplary embodiment, the user interface 160 may be operable to configure, manage and/or control operation of one or more components and/or modules in the X-ray imaging system 100. In this regard, the user interface 160 may be operable to configure, manage and/or control operation of the controller 150, user interface 160, display device 165, X-ray source 105, X-ray detector 107, C-arm gantry 110, C-arm carrier 111, upper ceiling mount 140, lower ceiling mount 146 and/or the cooling system 168. The user interface 160 may include hard button(s), soft button(s), rotary encoder(s), a touchscreen, motion tracking, voice recognition, a mousing device, keyboard, trackball, joystick(s), a touchpad, camera and/or any other device capable of receiving a user directive. In certain embodiments, one or more of the user interfaces 160 may be integrated into other components, such as the display device 165, for example. As an example, user interface 160 may include a touchscreen display. In some examples the user interface 160 may be remotely located relative to the X-ray imaging system 100. For example, the user interface 160 may be communicatively coupled to the controller 150 and/or the X-ray imaging system 100 via a wired or wireless connection.

As an example, a user of the X-ray imaging system 100 may input a desired isocenter position via the user interface 160. The controller 150 may then determine position adjustments to one or more of the C-arm gantry 110 and/or the upper ceiling mount 140 to align an isocenter of the X-ray imaging system 100 with the desired isocenter position. As another example, a user of the X-ray imaging system 100 may directly control the position of one or more components of the X-ray imaging system 100 relative to other components of the X-ray imaging system 100 via the user interface 160. For example, the user may directly input, via a joystick or knob, for example, position adjustments to one or more components of the X-ray imaging system 100. As another example, the motion of the components of the X-ray imaging system 100 may be pre-programmed such that the user does not directly control any movement, but instead initiates the start of the pre-programmed motion. The motion may include complex motions, with continuous motion of the isocenter.

The controller 150 is further communicatively coupled to a display device 165 for displaying one or more X-ray images acquired via the X-ray detector 107. Further, in some examples, one or more of the controller 150, the user interface 160, and the display device 165 may be positioned away from (e.g., remotely from) the remaining components of the X-ray imaging system 100. The display device 165 may be any device capable of communicating visual information to a user. For example, a display device 165 may include a liquid crystal display, a light emitting diode display, and/or any suitable display or displays.

The X-ray imaging system 100 may further include a cooling system 168 for cooling the X-ray source 105 and/or the X-ray detector 107. The cooling system 168 may include one or more flexible tubes and a pump, as an illustrative and non-limiting example, providing cooling fluid to the X-ray source 105 to transfer thermal energy away from the X-ray source 105. The cooling system 168 may actively cool the X-ray source 105 and the X-ray detector 107 independently, or in some examples may cool the X-ray detector 107 by any suitable type of derivation of the cooling circuit for the X-ray source 105.

Components of the X-ray imaging system 100 may be implemented in software, hardware, firmware, and/or the like. The various components of the X-ray imaging system 100 may be communicatively linked. Components of the X-ray imaging system 100 may be implemented separately and/or integrated in various forms. For example, the display device 165 and the user input device 130 may be integrated as a touchscreen display.

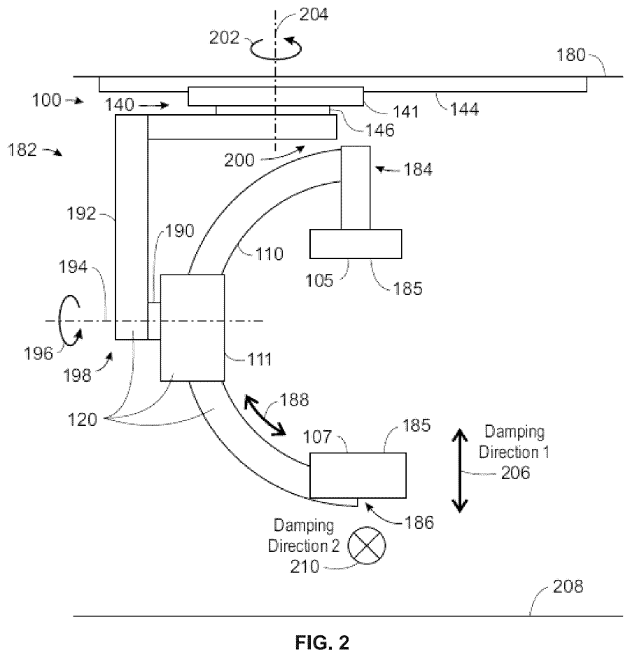

FIG. 2 is a schematic diagram of a side view of an exemplary X-ray imaging system 100 mounted to a ceiling 180, in accordance with various embodiments. The X-ray imaging system 100 of FIG. 2 shares various characteristics with the X-ray imaging system of FIG. 1 as described above. Referring to FIG. 2, the X-ray imaging system 100 is provided in an examination room 182 having a ceiling 180 and a floor 208. The X-ray imaging system 100 comprises an X-ray source 105, and X-ray detector 107, a gantry structure 120 having a C-arm gantry 110, a C-arm carrier 111 and a mounting structure 192, an upper ceiling mount 140, and a lower ceiling mount, among other things.

The gantry structure 120 comprises a C-arm gantry 110, a C-arm carrier 111, and a mounting structure 192. In certain embodiments, the C-arm gantry 110 is made of carbon fiber. The X-ray radiation source 105 is coupled to a first end 184 of the C-arm gantry 110 and the X-ray detector 107 is coupled to a second end 186 of the C-arm gantry 110 opposite the first end 184 (e.g., forming the image chain 185). The C-arm gantry 110 is coupled to the C-arm carrier 111 (e.g., C-arm rotation device), which is configured to rotate the C-arm gantry 110 in an orbital direction 188 relative to the C-arm carrier 111 about an isocenter of the X-ray radiation source 105 and the X-ray detector 107. The C-arm carrier 111 includes rollers (e.g., guiding rollers) to guide movement of the C-arm gantry 110 relative to the C-arm carrier 111.

The C-arm carrier 111 is coupled to a pivot 190 (e.g., pivot point or shaft). The pivot 190 is coupled to the mounting structure 192. The pivot 190 is driven by a motor (e.g., C-arm motor or any suitable motor) configured to rotate both the C-arm carrier 111 and the C-arm gantry 110 about a rotational axis 194 (e.g., horizontal axis) of the pivot 190 as indicated by arrow 196. In certain embodiments, the mounting structure 192 is an L-arm coupled to the upper ceiling mount 140 via the lower ceiling mount 146. The pivot 190 is coupled to a first end 198 of the L-arm 192 and the lower ceiling mount 146 is coupled to a second end 200 of the L-arm 192. The lower ceiling mount 146 is configured to couple the gantry structure 120 to the upper ceiling mount 140 (and, thus, mount the X-ray imaging system 100 to the ceiling 180). In certain embodiments, the L-arm may rotate about an end of the L-arm coupled to the upper ceiling mount 140 via the swivel bearing of the lower ceiling mount 146. In particular, the L-arm (and the gantry structure 120) rotate in direction 202 about a rotational axis 204 of the swivel bearing.

The lower ceiling mount 146 is coupled to the upper ceiling mount 140. In particular, a swivel bearing of the lower ceiling mount 146 is rotatably coupled to a chassis 141 of the upper ceiling mount 140. The chassis 141 (driven by a motor) is configured to move the X-ray imaging system 100 along the rail system 144 installed on the ceiling 180. In various embodiments, the lower ceiling mount 146 is configured to dampen vibrations caused by motion of the gantry structure 120 for an entirety of the gantry structure 120. For example, the movement of the gantry structure 120 may include the rotational movement of the C-arm gantry 110 in the orbital direction 188, rotational movement in the direction 196 about the axis 194 of the pivot 190 where the C-arm carrier 111 is coupled to an end of the mounting structure 192 (e.g., L-arm), and/or rotational movement in the direction 202 about the 204 axis of where the lower ceiling mount 146 (e.g., axis 204 of the swivel bearing) is coupled to the ceiling 180. The lower ceiling mount 146 is configured to dampen vibrations caused by movement of the gantry structure 120 for an entirety of the gantry structure 120 along the multiple directions. The multiple directions may include orthogonal directions. For example, the orthogonal directions may include a vertical direction 206 extending between a floor 208 and the ceiling 180 and a horizontal direction 210 (into the page in FIG. 2) that is both perpendicular to the vertical direction 206 and parallel with the floor 208.

The lower ceiling mount includes a swivel bearing (e.g., which couples the gantry structure to the chassis 141 of the upper ceiling mount 140 installed on the ceiling 180) and an imager mount. The swivel bearing is disposed on a side of the body facing the ceiling 180. The imager mount includes a body having a first end and a second end opposite the first end, a first mount connection disposed on the first end, and a second mount connection coupled to the second end. The imager mount 148 further comprises a safety hook that prevents the gantry structure 120 from falling if the first and/or second mount connections fail. The safety hook comprises two parallel vertical holders having a first end attached between the first end and the second end of the body, and a second end having an aperture. The two parallel vertical holders extend on opposite sides of a portion of the gantry structure 120 (e.g., a center rib of the L-arm 192). A shaft of the safety hook extends through the apertures in the second ends of the vertical holders and through a slot hole in the gantry structure 120. The shaft is secured to the vertical holders by a washer and nut, a clip ring, or any suitable attachment mechanism. The first mount connection and the second mount connection both directly contact both the mounting structure 192 of the gantry structure 120 and the body. In various embodiments, the safety hook directly contacts the body but does not receive a load of the gantry structure 120 in a normal condition. If the first and/or second mount connections fail, the safety hook receives the load of the gantry structure 120. In an exemplary embodiment, the first mount connection includes one or more mounting brackets, damping pads or dampers (e.g., made of elastomeric material such as rubber), and mounting screws and/or any suitable attachment mechanism. In certain embodiments, the second mount connection includes a shaft and bushings coupled to opposite ends of the shaft, and wherein the shaft extends through a portion of the gantry structure 120.

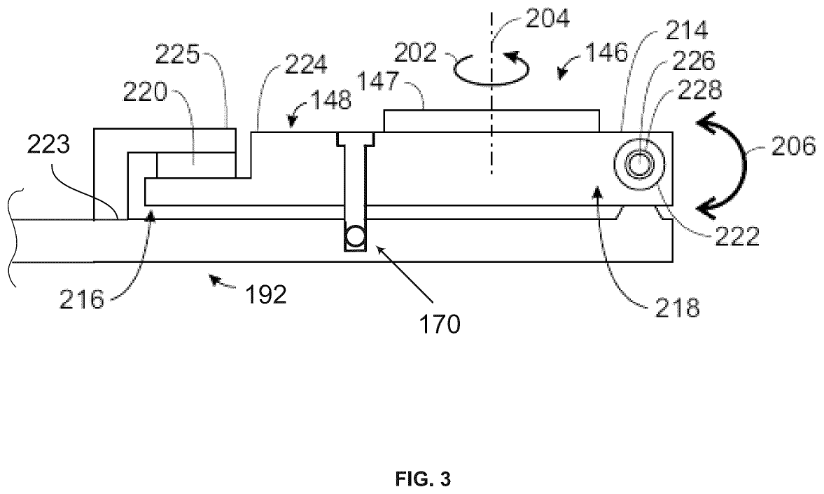

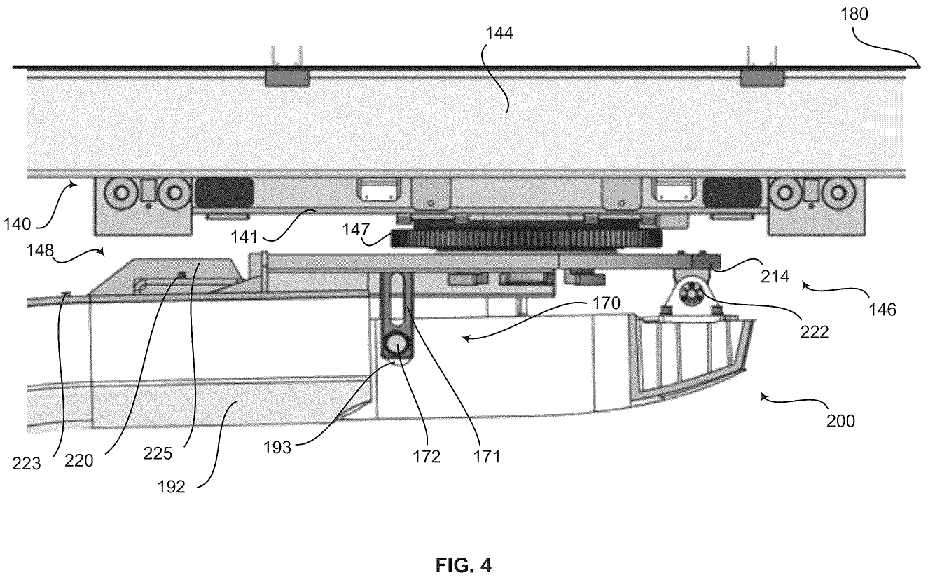

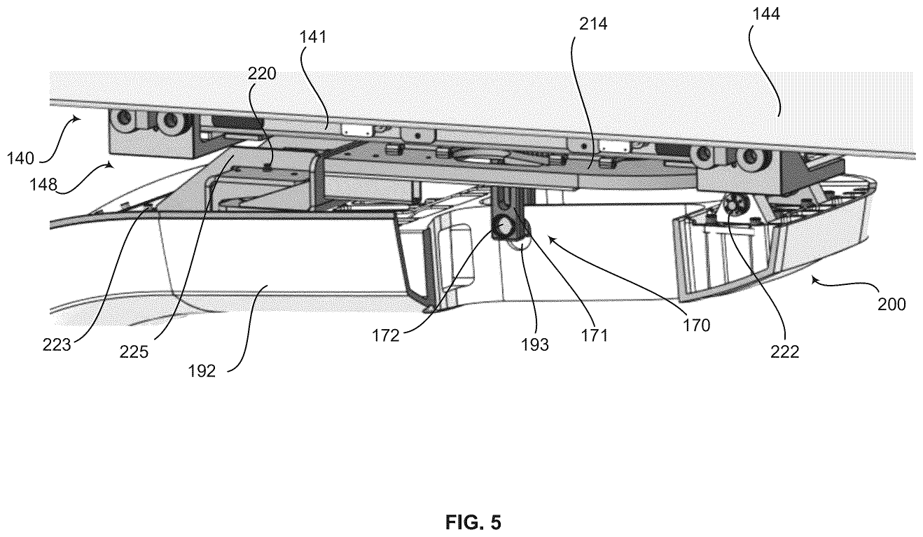

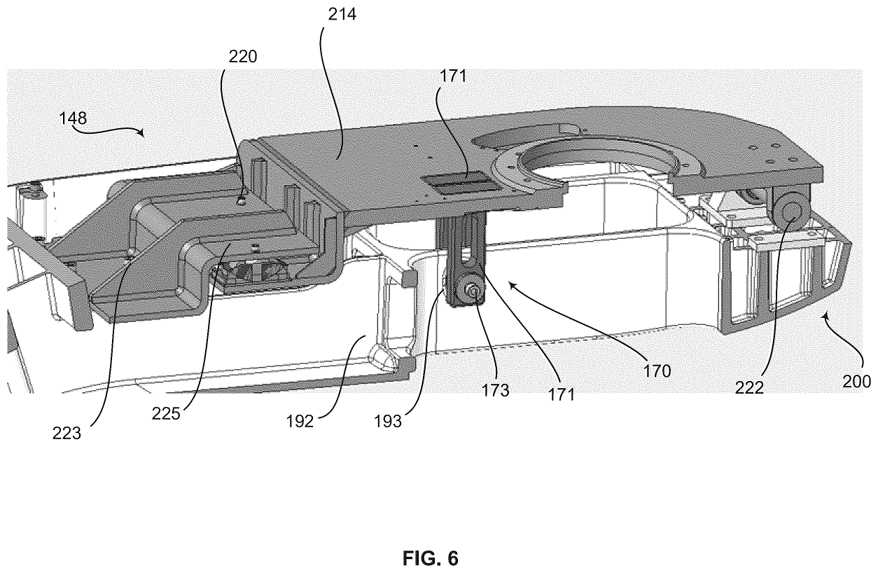

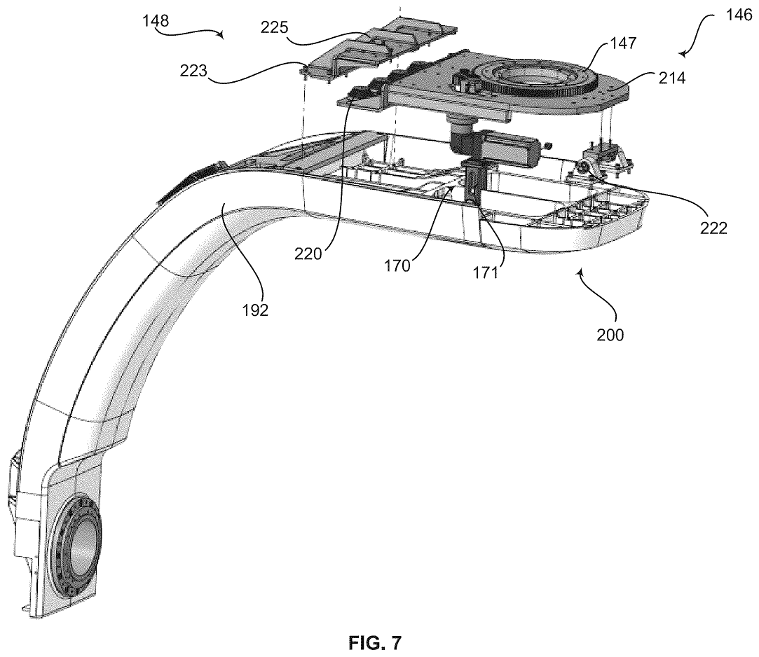

FIG. 3 is a schematic diagram of a lateral view of a lower ceiling mount 146 configured to pivotally mount an X-ray imaging system 100 to an upper ceiling mount 140 installed on a ceiling 180, in accordance with various embodiments. FIG. 4 is a schematic diagram of a lateral view of an X-ray imaging system mounted to a ceiling 180 by a lower ceiling mount 146 and an upper ceiling mount 140, in accordance with various embodiments. FIG. 5 is a schematic diagram of a perspective view of an X-ray imaging system 100 mounted to a ceiling 180 by a lower ceiling mount 146 and an upper ceiling mount 140, in accordance with various embodiments. FIG. 6 is a schematic diagram of a cross-sectional view of a lower ceiling mount 146 configured to pivotally mount an X-ray imaging system 100 to an upper ceiling mount 140 installed on a ceiling 180, in accordance with various embodiments. FIG. 7 is a schematic diagram of an exploded view of a lower ceiling mount 146 configured to pivotally mount an X-ray imaging system 100 to an upper ceiling mount 140 installed on a ceiling 180, in accordance with various embodiments. The upper ceiling mount 140 and lower ceiling mount 146 for mounting a mounting structure 192 (e.g., L-arm) of a medical imaging system (e.g., an X-ray system 100) to a ceiling 180 as shown in FIGS. 3-7 shares various characteristics with the upper ceiling mount 140 and lower ceiling mount 146 for mounting a mounting structure 192 (e.g., L-arm) of a medical imaging system (e.g., an X-ray system 100) to a ceiling 180 described above with reference to FIGS. 1 and 2.

Referring to FIGS. 3-7, a lower ceiling mount 146 is coupled to a mounting structure 192 of a medical imaging system, such as a gantry structure of an X-ray imaging system. Only a portion of the mounting structure 192 is shown in FIGS. 3-7. In various embodiments, the portion of the mounting structure 192 may be an L-arm 192 of a gantry structure 120 as shown in FIGS. 1-2. The lower ceiling mount 146 is configured to be rotatably coupled to an upper ceiling mount 140 installed on the ceiling 180. The upper ceiling mount 140 is configured to move (e.g., translocate) the medical imaging system (e.g., X-ray imaging system 100) from one location to another location (e.g., in a linear direction) on the ceiling 180. The upper ceiling mount 140 comprises a chassis 141, a rail system 144, and a motor. The rail system 144 comprises parallel rails coupled to the ceiling 180. The chassis 141 comprises linear guide assemblies having rollers that slidably couple the chassis 141 to protrusions extending from the rails of the rail system 144. The motor is operable to drive movement of the chassis 141 linearly along the rails to adjust a position of the medical imaging system (e.g., X-ray imaging system 100).

The lower ceiling mount 146 includes a swivel bearing 147 and an imager mount 148. A bottom side of the swivel bearing 147 is disposed on a top side 224 of a body 214 of the imager mount 148 facing the ceiling 180. A top side of the swivel bearing 147 may be rotatably coupled to the chassis 141 of the upper ceiling mount 140. For example, the swivel bearing 147 may comprise a fixed outer ring attached to the chassis 141 and a pivotable inner ring coupled to the imager mount 148. A motor may drive rotation of the inner ring of the swivel bearing 147 to adjust an orientation of the medical imaging system (e.g., X-ray imaging system 100) by rotating the imager mount 148 to a desired position. The swivel bearing 147 is configured to enable the medical imaging system (e.g., X-ray imaging system 100) to rotate 360 degrees in the direction 202 about the rotational axis 204 of the swivel bearing 147.

The imager mount 148 includes a body 214 having a first end 216 and a second end 218 opposite the first end 216, a first mount connection 220, 223, 225 disposed on the first end 216, a second mount connection 222 coupled to the second end 218, and a safety hook 170 attached to the body 214 between the first 216 and second 218 ends. As shown in FIGS. 3-6, both the first mount connection 220, 223, 225 and the second mount connection 222 directly contact both the mounting structure 192 and the body 214. In certain embodiments, the first mount connection 220, 223, 225 includes a mounting bracket 225 attached at a first end to the body 214 by attachment mechanisms 220, such as one or more damping pads or dampers (e.g., made of an elastomeric material such as rubber) and mounting screws or any suitable attachment mechanisms. A second end of the mounting bracket 225 is attached to the mounting structure 192 by attachment mechanisms 223, such as mounting screws or any suitable attachment mechanisms. The first mount connection 220, 223, 225 is disposed on a top side 224 of the body 214 (e.g., facing the ceiling 180). In particular, the first mount connection 220, 223, 225 is disposed between and contacts the mounting structure 192 and the body 214.

The second mount connection 222 includes a shaft 226 and bushings 228 coupled to opposite ends of the shaft 226. The shaft 226 extends through a portion of a second end 200 of the mounting structure 192 and portions of the body 214 located on a bottom side (e.g., facing away from the ceiling 180) of the body 214. In various embodiments, the body 214 is made of a material that is stiffer than both the first mount connection 220, 223, 225 and the second mount connection 222. The stiffer body 214 minimizes the movement of the gantry structure 120 during damping of vibrations.

The safety hook 170 is operable to prevent the medical imaging system (e.g., the X-ray imaging system 100) from falling from the ceiling 180 if the first 220, 223, 225 and/or second 222 mount connections fail. The safety hook 170 comprises two parallel vertical holders 171 having a first end attached between the first end 216 and the second end 218 of the body 214, and a second end having an aperture. The two parallel vertical holders 171 extend on opposite sides of the mounting structure 192 (e.g., a center rib of an L-arm 192 of a gantry structure 120). A shaft 172 of the safety hook 170 extends through the apertures in the second ends of the vertical holders 171 and through a slot hole 193 in the mounting structure 192. The shaft 172 may be a bolt or any suitable attachment mechanism. The shaft 172 is secured to the vertical holders 171 by a washer and nut 173, a clip ring, or any suitable attachment mechanism. The first mount connection 220, 223, 225 and the second mount connection 222 both directly contact the mounting structure 192 of the gantry structure 120 and the body 214. In various embodiments, the safety hook 170 directly contacts the body 214 at the first end of the safety hook 170 but does not receive a load of the medical imaging system in a normal condition. For example, the portion of the mounting structure 192 defining the slot hole 193 through which the shaft 172 of the safety hook 170 extends may not contact the shaft 172 of the safety hook 170 in the normal condition. If the first 220, 223, 225 and/or second 222 mount connections fail, the shaft 172 of the safety hook 170 catches the mounting structure 192 and receives the load of the medical imaging system (e.g., the gantry structure 120 of the X-ray imaging system 100). The safety hook 170 may comprise steel and/or any suitable high strength metal. In various embodiments, interior portions of the two parallel vertical holders 171 may be removed to reduce the weight and/or cost of the safety hook. The first ends of the two parallel vertical holders 171 may be fixedly attached to the body 214 by screws, welding, and/or any suitable attachment mechanism.

In various embodiment, the lower ceiling mount 146 is configured to dampen vibrations caused by motion of the medical imaging system (e.g., a gantry structure 120 of an X-ray imaging system 100) for an entirety of the medical imaging system along the multiple directions. The multiple directions may include orthogonal directions. For example, the orthogonal directions may include a vertical direction 206 (damping direction 1) extending between the floor and the ceiling 180 and the horizontal direction 210 (damping direction 2) that is both perpendicular to the vertical direction 206 and parallel with the floor 208 as depicted in FIG. 2. As depicted in FIGS. 2 and 3, the damping directions 1 and 2 may include a circumferential element, such as about a rotational axis of the shaft 226 and side to side movement of lateral sides of the lower ceiling mount 146, respectively.

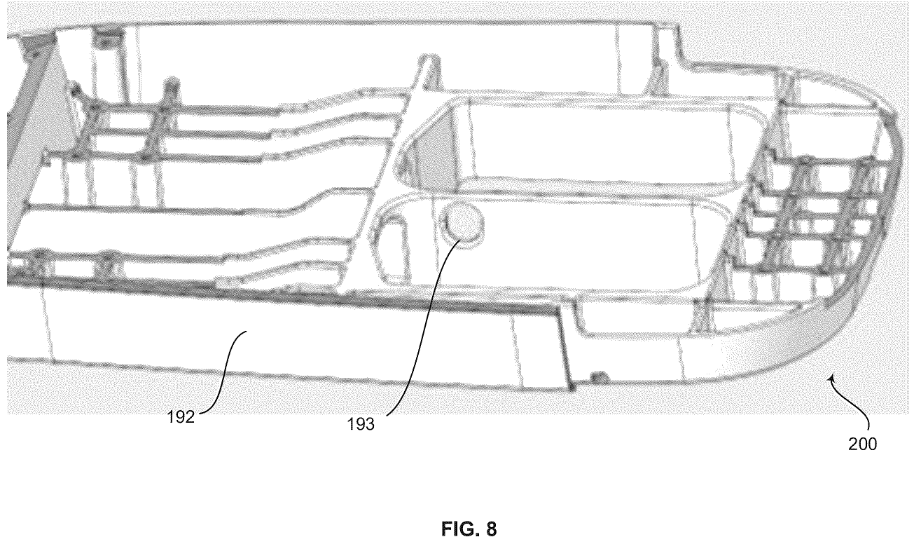

FIG. 8 is a schematic diagram of a perspective view of a portion 192 of an X-ray imaging system having a slot hole 193 for receiving a safety hook, in accordance with various embodiments. Referring to FIG. 8, a portion of a mounting structure 192 of a medial imaging system (e.g., an X-ray imaging system 100) is shown. The mounting structure 192 shares various characteristics with the mounting structure 192 discussed above with respect to FIGS. 1-7. The mounting structure 192 may be an L-arm having a first end 198 coupled to a C-arm carrier 111 by a pivot 190 and a second end 200 coupled to a lower ceiling mount 146 as described above with respect to FIG. 2. In various embodiments, the mounting structure 192 may be carbon fiber, a high strength metal such as steel, or any suitable high strength material. In an exemplary embodiment, the mounting structure 192 comprises a slot hole 193 for receiving a safety hook 170 as shown in FIGS. 3-7. For example, as explained with reference to FIGS. 3-7, the safety hook 170 may comprise two parallel vertical holders 171 that extend on opposite sides of the mounting structure 192, such as a center rib of an L-arm 192 of a gantry structure 120. A shaft 172 of the safety hook 170 may extend through the apertures in the second ends of the vertical holders 171 and through a slot hole 193 in the mounting structure 192. In various embodiments, the safety hook 170 does not receive a load of the medical imaging system in a normal condition. For example, the portion of the mounting structure 192 defining the slot hole 193 through which the shaft 172 of the safety hook 170 extends may not contact the shaft 172 of the safety hook 170 in the normal condition. Instead, the shaft 172 of the safety hook 170 may contact the portion of the mounting structure 192 that defines the slot hole 193 such that the safety hook 170 carries the load of the medical imaging system (e.g., the gantry structure 120 of the X-ray imaging system 100) if primary connections between the medical imaging system fail. In this way, the safety hook 170 is operable to prevent the medical imaging system (e.g., the X-ray imaging system 100) from falling from the ceiling 180 if the primary mount connections fail.

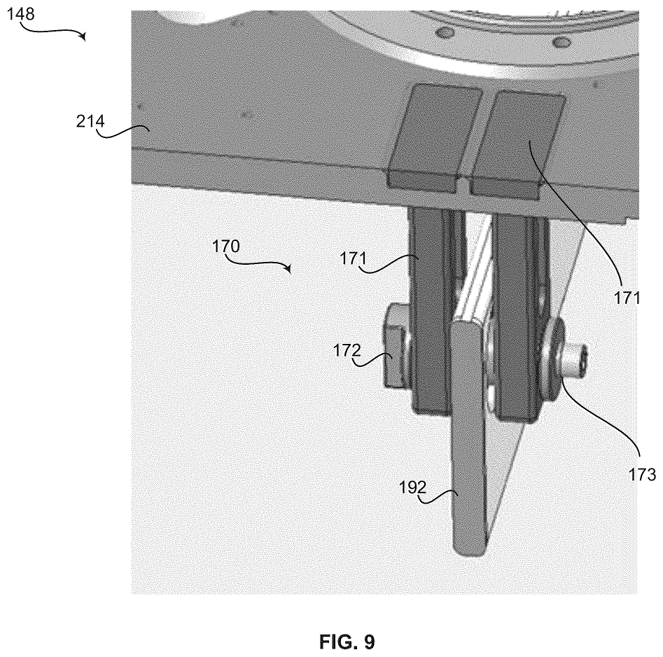

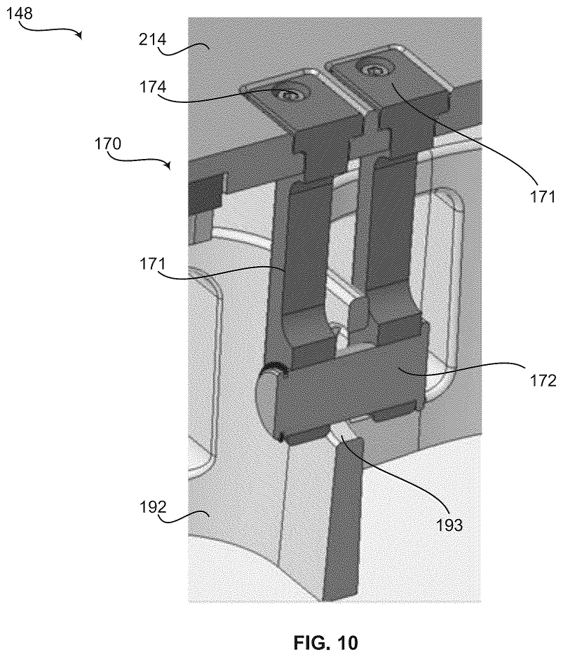

FIG. 9 is a schematic diagram of a perspective view of a safety hook 170 provided between a lower ceiling mount and an X-ray imaging system, in accordance with various embodiments. FIG. 10 is a schematic diagram of a cross-sectional view of a safety hook 170 provided between a lower ceiling mount and an X-ray imaging system, in accordance with various embodiments. Referring to FIGS. 9-10, a safety hook 170 is shown fixedly attached to a body 214 of an imager mount 148 of a lower ceiling mount at a first (e.g., top) end and extending through a slot hole 193 in a mounting structure 192 of a medical imaging system (e.g., the X-ray imaging system 100). The safety hook 170 comprises two parallel vertical holders 171 having a first end attached to a body 214 of a lower ceiling mount, and a second end having an aperture. The two parallel vertical holders 171 extend on opposite sides of the mounting structure 192 (e.g., a center rib of an L-arm 192 of a gantry structure 120). A shaft 172 of the safety hook 170 extends through the apertures in the second ends of the vertical holders 171 and through the slot hole 193 in the mounting structure 192. The shaft 172 may be a bolt or any suitable attachment mechanism. The shaft 172 is secured to the vertical holders 171 by a washer and nut 173, a clip ring, or any suitable attachment mechanism. The safety hook 170 is securely attached to the body 214 of the imager mount 148 by welding, mounting screws 174, and/or any suitable attachment mechanism. The safety hook 170 directly contacts the body 214 at the first end of the safety hook 170 but does not receive a load of the medical imaging system in a normal condition. For example, the portion of the mounting structure 192 defining the slot hole 193 through which the shaft 172 of the safety hook 170 extends may not contact the shaft 172 of the safety hook 170 in the normal condition (i.e., non-failure condition). If the mount connections of the imager mount 148 fail (i.e., failure condition), the shaft 172 of the safety hook 170 catches the falling mounting structure 192 and receives the load of the medical imaging system (e.g., the gantry structure 120 of the X-ray imaging system 100). The safety hook 170 may comprise steel and/or any suitable high strength metal. In various embodiments, interior portions of the two parallel vertical holders 171 are removed to reduce the weight and/or cost of the safety hook 170. In operation, the safety hook 170 is operable to prevent the medical imaging system (e.g., the X-ray imaging system 100) from falling from the ceiling 180 if the mount connections of the ceiling mount fail.

Although various embodiments described above provide an X-ray imaging system 100 mounted to a ceiling 180 by and upper ceiling mount 140 and a lower ceiling mount 146 comprising a safety hook 170, unless so claimed the safety hook 170 is not limited to use in ceiling mounts 140, 146 for X-ray imaging systems 100, and may be provided in ceiling mounts 140, 146 of any suitable medical imaging systems.

Aspects of the present disclosure provide a safety hook 170 of a medical imaging system ceiling mount assembly 140, 146 operable to receive a load of a medical imaging system 100 in a failure condition of the medical imaging system ceiling mount assembly 140, 146. The safety hook may comprise two parallel vertical holders 171 and a shaft 172. Each of the two parallel vertical holders 171 may comprise a first end operable to be attached to a body 214 of the medical imaging system ceiling mount assembly 140, 146. Each of the two parallel vertical holders 171 may comprise a second end opposite the first end, the second end comprising an aperture. A first of the two parallel vertical holders 171 may be configured to be positioned adjacent a first side of a mounting structure 192 of the medical imaging system 100. A second of the two parallel vertical holders 171 may be configured to be positioned adjacent a second side of the mounting structure 192 of the medical imaging system 100. The shaft 172 may extend through each of the aperture at the second end of the first of the two parallel vertical holders 171, a slot hole 193 in the mounting structure 192 of the medical imaging system 100, and the aperture at the second end of the second of the two parallel vertical holders 171.

In a representative embodiment, the body 214 of the medical imaging system ceiling mount assembly 140, 146 may comprise mount connections 220, 222, 223, 225 configured to receive the medical imaging system 100 in a normal condition. The normal condition corresponds with a normal connection between the medical imaging system 100 and the medical imaging system mount assembly 140, 146 by the mount connections 220, 222, 223, 225. The failure condition may correspond with a failed connection between the medical imaging system 100 and the medical imaging system mount assembly 140, 146 by one or more of the mount connections 220, 222, 223, 225. In an exemplary embodiment, the safety hook 170 does not receive the load of the medical imaging system 100 in the normal condition. In various embodiments, the safety hook 170 comprises steel. In certain embodiments, the safety hook 170 comprises a nut and washer 173 operable to secure the shaft 172 in the aperture at the second end of each of the two parallel vertical holders 171. The shaft 172 may be threaded. In a representative embodiment, the safety hook 170 comprises a clip ring 173 operable to secure the shaft 172 in the aperture at the second end of each of the two parallel vertical holders 171. In an exemplary embodiment, the first end of each of the two parallel vertical holders 171 is configured to be attached to the body 214 of the medical imaging system ceiling mount assembly 140, 146 by either welding or mounting screws 174. In certain embodiments, an interior portion of a central part of each of the two parallel vertical holders 171 between the first end and the second end of each of the two parallel vertical holders 171 is void of material.

Various embodiments provide a ceiling mount assembly 140, 146 operable to mount a medical imaging system 100 to a ceiling 180. In accordance with various embodiments, the ceiling mount assembly 140, 146 comprises a body 214, mount connections 220, 222, 223, 225, and a safety hook 170. The body 214 may be configured to be coupled to the ceiling 180. The mount connections may be configured to couple the body 214 to the medical imaging system 100 to support a load of the medical imaging system 100 in a normal condition. The safety hook 170 may comprise two parallel vertical holders 171 and a shaft 172. Each of the two parallel vertical holders may comprise a first end attached to the body 214. Each of the two parallel vertical holders may comprise a second end opposite the first end, the second end comprising an aperture. The two parallel vertical holders 171 may be configured to extend across opposite sides of a mounting structure 192 of the medical imaging system 100. The shaft 172 of the safety hook 170 may extend through the aperture of each of the two parallel vertical holders 171 and a slot hole 193 in the mounting structure 192 of the medical imaging system 100. The safety hook 170 may be operable to receive the load of the medical imaging system 100 in a failure condition of one or more of the mount connections 220, 222, 223, 225.

In an exemplary embodiment, the mount connections 220, 222, 223, 225 comprise a first mount connection 220, 223, 225 at a first end of the body and a second mount connection 222 at a second end of the body 214 opposite the first end of the body 214. The safety hook 170 may be provided between the first mount connection 220, 223, 225 and the second mount connection 222. In various embodiments, one or more of the mount connections 220, 222, 223, 225 comprises a damper 220 configured to dampen vibrations caused by movement of the medical imaging system 100. In certain embodiments, the safety hook 170 does not receive the load of the medical imaging system 100 in the normal condition. In a representative embodiment, the safety hook 170 comprises steel. In an exemplary embodiment, the shaft 172 of the safety hook 170 is secured in the aperture at the second end of each of the two parallel vertical holders 171 of the safety hook 170 by a clip ring or nut with washer 173. In various embodiments, the first end of each of the two parallel vertical holders 171 is attached to the body 214 by either welding or mounting screws 174.

Certain embodiments provide a ceiling mounted medical imaging system 100 comprising a medical imaging system 100 and a ceiling mouth assembly 140, 146. The medical imaging system 100 may be operable to acquire medical image data. The medical imaging system 100 may comprise a mounting structure 192 having a slot hole 193. The ceiling mount assembly 140, 146 may comprise a body 214, mount connections 220, 222, 223, 225, and a safety hook 170. The body 214 may be configured to be coupled to a ceiling 180. The body 214 may be linearly movable and rotatable with respect to the ceiling 180. The mount connections 220, 222, 223, 225 may be configured to couple the body 214 to the mounting structure 192 of the medical imaging system 100 to support a load of the medical imaging system 100 in a normal condition. The safety hook 170 may comprise two parallel vertical holders 171 and a shaft 172. Each of the two parallel vertical holders 171 may comprise a first end attached to the body 214. Each of the two parallel vertical holders 171 may comprise a second end opposite the first end, the second end comprising an aperture. The two parallel vertical holders 171 may be configured to extend on opposite sides of the mounting structure 192 of the medical imaging system 100. The shaft 172 may extend through the aperture of each of the two parallel vertical holders 171 and the slot hole 193 in the mounting structure 192 of the medical imaging system 100. The safety hook 170 may be operable to receive the load of the medical imaging system 100 in a failure condition of one or more of the mount connections 220, 222, 223, 225.

In various embodiments, the ceiling mounted medical imaging system 100 is an X-ray medical imaging system 100. In certain embodiments, the safety hook 170 does not receive the load of the medical imaging system 100 in the normal condition. In a representative embodiment, the shaft 172 of the safety hook 170 is secured in the aperture at the second end of each of the two parallel vertical holders 171 of the safety hook 170 by a clip ring or nut with washer 173. In an exemplary embodiment, the first end of each of the two parallel vertical holders 171 is attached to the body 214 by either welding or mounting screws 174.

As utilized herein the term “circuitry” refers to physical electronic components (i.e. hardware) and any software and/or firmware (“code”) which may configure the hardware, be executed by the hardware, and or otherwise be associated with the hardware. As used herein, for example, a particular processor and memory may comprise a first “circuit” when executing a first one or more lines of code and may comprise a second “circuit” when executing a second one or more lines of code.

As utilized herein, “and/or” means any one or more of the items in the list joined by “and/or”. As an example, “x and/or y” means any element of the three-element set {(x), (y), (x, y)}. As another example, “x, y, and/or z” means any element of the seven-element set {(x), (y), (z), (x, y), (x, z), (y, z), (x, y, z)}. As utilized herein, the term “exemplary” means serving as a non-limiting example, instance, or illustration. As utilized herein, the terms “e.g.,” and “for example” set off lists of one or more non-limiting examples, instances, or illustrations. As utilized herein, a component is “operable” or “configured” to perform a function whenever the component comprises the necessary structure to perform the function, regardless of whether the function is performed.

While the present disclosure has been described with reference to certain embodiments, it will be understood by those skilled in the art that various changes may be made and equivalents may be substituted without departing from the scope of the present disclosure. In addition, many modifications may be made to adapt a particular situation or material to the teachings of the present disclosure without departing from its scope. Therefore, it is intended that the present disclosure not be limited to the particular embodiment disclosed, but that the present disclosure will include all embodiments falling within the scope of the appended claims.

What is claimed is:

1. A safety hook of a medical imaging system ceiling mount assembly operable to receive a load of a medical imaging system in a failure condition of the medical imaging system ceiling mount assembly, the safety hook comprising:
two parallel vertical holders, each of the two parallel vertical holders comprising:
a first end operable to be attached to a body of the medical imaging system ceiling mount assembly; and
a second end opposite the first end, the second end comprising an aperture,
wherein a first of the two parallel vertical holders is configured to be positioned adjacent a first side of a mounting structure of the medical imaging system, and a second of the two parallel vertical holders is configured to be positioned adjacent a second side of the mounting structure of the medical imaging system; and
a shaft extending through each of:
the aperture at the second end of the first of the two parallel vertical holders;
a slot hole in the mounting structure of the medical imaging system; and
the aperture at the second end of the second of the two parallel vertical holders.

2. The safety hook of claim 1, wherein:
the body of the medical imaging system ceiling mount assembly comprises mount connections configured to receive the medical imaging system in a normal condition;
the normal condition corresponds with normal connections between the medical imaging system and the medical imaging system mount assembly by the mount connections; and
the failure condition corresponds with a failed connection between the medical imaging system and the medical imaging system mount assembly by one or more of the mount connections.

3. The safety hook of claim 2, wherein the safety hook does not receive the load of the medical imaging system in the normal condition.

4. The safety hook of claim 1, wherein the safety hook comprises steel.

5. The safety hook of claim 1, comprising a nut and washer operable to secure the shaft in the aperture at the second end of each of the two parallel vertical holders, wherein the shaft is threaded.

6. The safety hook of claim 1, comprising a clip ring operable to secure the shaft in the aperture at the second end of each of the two parallel vertical holders.

7. The safety hook of claim 1, wherein the first end of each of the two parallel vertical holders is configured to be attached to the body of the medical imaging system ceiling mount assembly by either welding or mounting screws.

8. The safety hook of claim 1, wherein an interior portion of a central part of each of the two parallel vertical holders between the first end and the second end of each of the two parallel vertical holders is void of material.

9. A ceiling mount assembly operable to mount a medical imaging system to a ceiling, the ceiling mount assembly comprising:
a body configured to be coupled to the ceiling;
mount connections configured to couple the body to the medical imaging system to support a load of the medical imaging system in a normal condition; and
a safety hook comprising:
two parallel vertical holders, each of the two parallel vertical holders comprising:
a first end attached to the body; and
a second end opposite the first end, the second end comprising an aperture,
wherein the two parallel vertical holders are configured to extend across opposite sides of a mounting structure of the medical imaging system; and
a shaft extending through the aperture of each of the two parallel vertical holders and a slot hole in the mounting structure of the medical imaging system,
wherein the safety hook is operable to receive the load of the medical imaging system in a failure condition of one or more of the mount connections.

10. The ceiling mount assembly of claim 9, wherein the mount connections comprise a first mount connection at a first end of the body and a second mount connection at a second end of the body opposite the first end of the body, and wherein the safety hook is provided between the first mount connection and the second mount connection.

11. The ceiling mount assembly of claim 10, wherein one or more of the mount connections comprises a damper configured to dampen vibrations caused by movement of the medical imaging system.

12. The ceiling mount assembly of claim 9, wherein the safety hook does not receive the load of the medical imaging system in the normal condition.

13. The ceiling mount assembly of claim 9, wherein the safety hook comprises steel.

14. The ceiling mount assembly of claim 9, wherein the shaft of the safety hook is secured in the aperture at the second end of each of the two parallel vertical holders of the safety hook by a clip ring or nut with washer.

15. The ceiling mount assembly of claim 9, wherein the first end of each of the two parallel vertical holders is attached to the body by either welding or mounting screws.

16. A ceiling mounted medical imaging system, comprising:
a medical imaging system operable to acquire medical image data, the medical imaging system comprising a mounting structure having a slot hole; and
a ceiling mount assembly comprising:
a body configured to be coupled to a ceiling, wherein the body is linearly movable and rotatable with respect to the ceiling;
mount connections configured to couple the body to the mounting structure of the medical imaging system to support a load of the medical imaging system in a normal condition; and
a safety hook comprising:
two parallel vertical holders, each of the two parallel vertical holders comprising:
a first end attached to the body; and
a second end opposite the first end, the second end comprising an aperture,
wherein the two parallel vertical holders are configured to extend on opposite sides of the mounting structure of the medical imaging system; and
a shaft extending through the aperture of each of the two parallel vertical holders and the slot hole in the mounting structure of the medical imaging system,
wherein the safety hook is operable to receive the load of the medical imaging system in a failure condition of one or more of the mount connections.

17. The ceiling mounted medical imaging system of claim 16, wherein the ceiling mounted medical imaging system is an X-ray medical imaging system.

18. The ceiling mounted medical imaging system of claim 16, wherein the safety hook does not receive the load of the medical imaging system in the normal condition.

19. The ceiling mounted medical imaging system of claim 16, wherein the shaft of the safety hook is secured in the aperture at the second end of each of the two parallel vertical holders of the safety hook by a clip ring or nut with washer.

20. The ceiling mounted medical imaging system of claim 16, wherein the first end of each of the two parallel vertical holders is attached to the body by either welding or mounting screws.
